(19) United States
(12) Patent Application Publication
(10) Pub. No.: US 2020/0397738 A1
(43) Pub. Date: Dec. 24, 2020

(54) METHODS FOR TREATING CANCER AND PROTECTING RENEWABLE TISSUES

(71) Applicants: President and Fellows of Harvard College, Cambridge, MA (US); Shanghai Jiao Tong University School of Medicine, Shanghai (CN)

(72) Inventors: Zhi-Min YUAN, Newton, MA (US); Xixiang TANG, Xiamen (CN); Weiqi ZENG, Boston, MA (US)

(21) Appl. No.: 16/975,957

(22) PCT Filed: Feb. 27, 2018
(86) PCT No.: PCT/CN2018/077395

§ 371 Date: Aug. 26, 2020

(51) Int. Cl. A61K 31/341; A61K 31/704; A61K 31/513; A61K 33/243; A61K 31/337; A61K 31/475; A61K 38/14; A61K 38/15; A61P 35/00

(52) U.S. Cl. CPC A61K 31/341; A61K 31/704; A61K 31/513; A61K 33/243; A61P 35/00; A61K 31/475; A61K 38/14; A61K 38/15; A61K 31/337

(57) ABSTRACT

Provided are methods for treating a subject having or at risk of developing cancer by administering a compound that preserves p53. Also disclosed are methods for protecting renewable tissues by administering a compound that stabilizes EZH2.

### STATEMENT AS TO FEDERALLY FUNDED RESEARCH

This invention was made with government support under grant number 5R01CA183074 awarded by the National Institutes for Health. The government has certain rights in this invention.

### BACKGROUND OF THE INVENTION

Cancer is one of the deadliest threats to human health. In 2013, the global cancer burden was estimated to be at least 14.1 million new cases and 8.2 million cancer deaths. With these statistics predicted to increase further by 2025, a balanced approach to prevention, early detection and treatment is required. While many cancer therapies show promising clinical efficacy, severe toxic side effects limit their use as effective treatment options. Radiation and chemotherapeutic drugs that are routinely used in cancer therapy often work by inducing DNA damage, resulting in senescence and death of cancer cells. Unfortunately, the DNA damaging property of radiation and chemotherapeutic agents can also cause considerable damage and death of non-cancerous renewable tissues in patients receiving the therapy. Hence, a well-adjusted methodology needs to be developed that will effectively target cancer tissues and cells, while protecting non-cancerous, renewable tissues from DNA damage.

### SUMMARY OF THE INVENTION

The present invention provides methods for preventing and treating cancer, and protecting non-cancerous renewable tissues.

In a first aspect, the invention provides a method of preserving p53 in a cell without activation by contacting the cell with an effective amount of a compound that reduces the amount of MDM2/MDMX complex.

In another aspect, the invention provides a method of preserving p53 in a subject without activation by administering to the subject an effective amount of a compound that reduces the amount of MDM2/MDMX complex.

In another aspect, the invention provides a method of treating a subject with cancer by administering to the subject an effective amount of a compound that reduces the amount of MDM2/MDMX complex, wherein the reduction of the MDM2/MDMX complex results in preservation of p53 without its activation.

In some embodiments of the above aspect, the compound decreases tumor volume. In some embodiments of the above aspect, the compound decreases tumor or cancer cell growth. In some embodiments of the above aspect, the compound decreases tumor or cancer cell proliferation. In some embodiments of the above aspect, the compound preserves tumor or cancer cell p53 activity. In some embodiments of the above aspect, the compound preserves tumor or cancer cell p53 expression.

In some embodiments of the above aspect, the compound prevents tumorigenesis. In some embodiments of the above aspect, the compound induces tumor regression. In some embodiments of the above aspect, the compound improves survival.

In some embodiments of any of the above aspects, the compound does not induce apoptosis.

In some embodiments of any of the above aspects, p53 is preserved without its transcriptional activation. In some embodiments of any of the above aspects, p53 is preserved without its systemic activation.

In some embodiments of any of the above aspects, the compound preserves p53 by reducing the amount of MDM2/MDMX complex. In some embodiments of any of the above aspects, the reduction in the amount of MDM2/MDMX complex results from dissociation of the MDM2/MDMX complex by the said compound.

In some embodiments of the above aspect, the method further includes the step of administering a second therapeutic agent. In some embodiments of the above aspect, the second therapeutic agent is an anti-cancer agent.

In some embodiments of the above aspect, the anti-cancer agent is a chemotherapeutic agent. In some embodiments of the above aspect, the chemotherapeutic agent is an anthracycline (e.g., doxorubicin). In some embodiments of the above aspect, the chemotherapeutic agent is a nucleoside analog (e.g., fluorouracil). In some embodiments of the above aspect, the chemotherapeutic agent is a platinum-based anti-neoplastic agent (e.g., cisplatin). In some embodiments of the above aspect, the chemotherapeutic agent is a taxane (e.g., paclitaxel). In some embodiments of the above aspect, the chemotherapeutic agent is a vinca alkaloid (e.g., vincristine). In some embodiments of the above aspect, the chemotherapeutic agent is a glycopeptide antibiotic (e.g., bleomycin). In some embodiments of the above aspect, the chemotherapeutic agent is a polypeptide antibiotic (e.g., actinomycin D).

In some embodiments of the above aspect, the anti-cancer agent is a targeted therapeutic agent. In some embodiments of the above aspect, the targeted therapeutic agent is one or more of a tyrosine kinase inhibitor, a PI3K inhibitor, a multi-kinase inhibitor, a CDK4/6 inhibitor, an mTOR inhibitor, a NOTCH inhibitor, an HSP90 inhibitor, an HSP70 inhibitor, a proteasome inhibitor, or a tumor metabolism inhibitor.

In some embodiments of the above aspect, the anti-cancer agent is an immunotherapeutic agent. In some embodiments of the above aspect, the immunotherapeutic agent is one or more of an immune checkpoint inhibitor, a monoclonal antibody, a cancer vaccine, an antibody-drug conjugate, or a non-specific immunotherapeutic agent. In some embodiments of the above aspect, the immune checkpoint inhibitor is one or more of an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

In some embodiments of the above aspect, the compound is administered to the subject in an amount sufficient to treat the cancer or tumor. In some embodiments of the above aspect, the compound is administered to the subject in an amount sufficient to cause remission. In some embodiments of the above aspect, the compound is administered to the subject in an amount sufficient to reduce tumor volume. In some embodiments of the above aspect, the compound is administered to the subject in an amount sufficient to reduce tumor or cancer cell growth. In some embodiments of the above aspect, the compound is administered to the subject in an amount sufficient to reduce tumor or cancer cell proliferation. In some embodiments of the above aspect, the compound is administered to the subject in an amount sufficient to preserve tumor or cancer cell p53 activity. In some embodiments of the above aspect, the compound is administered to the subject in an amount sufficient to preserve tumor or cancer cell p53 expression. In some embodiments of the above aspect, the compound is administered to the subject in an amount sufficient to improve survival.

In some embodiments of the above aspect, the cancer is a p53-associated cancer.

In another aspect, the invention provides a method of preventing the development of cancer in a subject by: (i) identifying a subject at the risk of developing cancer, and (ii) administering to the subject an effective amount of a compound that reduces the amount of MDM2/MDMX complex, wherein the reduction of the MDM2/MDMX complex results in preservation of p53 without its activation.

In some embodiments of the above aspect, p53 is preserved without its transcriptional activation. In some embodiments of the above aspect, p53 is preserved without its systemic activation.

In some embodiments of the above aspect, the compound preserves p53 by reducing the amount of MDM2/MDMX complex. In some embodiments of the above aspect, the reduction in the amount of MDM2/MDMX complex results from dissociation of the MDM2/MDMX complex by the said compound.

In some embodiments of the above aspect, the subject at the risk of developing cancer is a subject with reduced p53 expression or activity but does not have a p53 gene mutation. In some embodiments of the above aspect, the subject at the risk of developing cancer is a subject with one or more inactivating p53 mutation.

In some embodiments of the above aspect, the cancer is a p53-associated cancer.

In another aspect, the invention provides a method for stabilizing EZH2 in renewable tissue by contacting the renewable tissue with an effective amount of a compound that reduces the amount of MDM2/MDMX complex.

In another aspect, the invention provides a method for stabilizing EZH2 in renewable tissue in a subject by administering to the subject an effective amount of a compound that reduces the amount of MDM2/MDMX complex.

In some embodiments of the above aspects, the compound stabilizes EZH2 by reducing the amount of MDM2/MDMX complex. In some embodiments of the above aspects, the reduction in the amount of MDM2/MDMX complex results from dissociation of the MDM2/MDMX complex by the said compound.

In some embodiments of the aforementioned aspects of the invention, the stabilized EZH2 protects the renewable tissue from DNA damage. In some embodiments of the above aspects, the renewable tissue is bone marrow, spleen, thymus, duodenum or any other tissue that is highly sensitive to damage.

In some embodiments of the above aspects of the invention, the DNA damage is caused by radiation or chemotherapeutic agents.

In some embodiments of the above aspect, the subject has cancer (e.g., a cancer patient). In some embodiments of the above aspect, the subject is being treated with radiation or chemotherapeutic agents (e.g., a cancer patient who is being exposed to radiation or chemotherapeutic drugs as part of the cancer treatment regimen that leads to collateral DNA damage of normal renewable tissues).

### Definitions

As used herein, the term “administering” refers to the act of providing or giving a subject a therapeutic agent (e.g., a compound to preserve p53), by any effective route. Exemplary routes of administration are described herein below.

As used herein, the term “apoptosis” refers to a process of programmed cell death that occurs in multicellular organisms. Apoptosis is a highly regulated process that can be initiated through one of two pathways, the intrinsic pathway in which the cell kills itself because it senses cell stress, or the extrinsic pathway in which the cell kills itself because of signals from other cells. In both the pathways, cell death is induced by activating caspases, which are proteases, or enzymes that degrade proteins.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be achieved by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “damage” and “DNA damage” refer to irradiation- or chemotherapeutic agent-mediated damage to the genomic DNA that can often lead to senescence and cell death.

As used herein, the terms “effective amount,” “therapeutically effective amount,” and a “sufficient amount” of a composition described herein refer to a quantity sufficient to, when administered to a subject, including a mammal (e.g., a human), cause beneficial or desired results, including effects at the cellular level, tissue level, or clinical results, and, as such, an “effective amount” or synonym thereto depends upon the context in which it is being applied. For example, in the context of treating cancer it is an amount of the composition sufficient to achieve a treatment response as compared to the response obtained without administration of the composition. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition of the present disclosure is an amount that results in a beneficial or desired result in a subject as compared to a control. As defined herein, a therapeutically effective amount of a composition of the present disclosure may be readily determined by one of ordinary skill by routine methods known in the art. Dosage regimen may be adjusted to provide the optimum therapeutic response.

As used herein, the terms “increasing” and “decreasing” refer to modulating resulting in, respectively, greater or lesser amounts, of function, expression, or activity of a metric relative to a reference. For example, subsequent to administration of a compound for preserving p53 in a method described herein, the amount of a marker of a metric (e.g., proliferation of cancer cells) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun. The term “reducing” is used interchangeably with the term “decreasing” herein.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “preserved” refers to maintaining a gene or a protein in its original or physiological state. For example, subsequent to administration of a compound for preserving p53 in a method described herein, the expression and/or activity of p53 will be modulated such that the expression and/or activity of p53 will not be higher (e.g., as is observed subsequent to treatment with p53 activating compounds), or lower (e.g., as is observed in p53-associated cancers), but be equal, similar, or comparable to p53 expression and/or activity observed under normal, physiological conditions (e.g., in a healthy individual without cancer).

As used herein, the term “p53-preserving” refers to those compounds, materials, compositions and/or dosage forms, which are capable of preserving or maintaining the expression and/or activity of p53 at its basal physiological level. For example, the p53-preserving compound (e.g., Compound 1) described herein preserves the activity and/or expression of p53 at the basal physiological level without activating, or inhibiting it.

As used herein, “preserving p53 without activation” refers to maintaining p53 in its basal physiological level without substantial activation or induction of its expression and/or activity. For example, subsequent to administration of the p53-preserving compound in the methods described herein, the expression and/or activity of p53 will be modulated such that the expression and/or activity of p53 will not be higher (e.g., as is observed subsequent to treatment with p53 activating compounds), but be equal, similar, or comparable to p53 expression and/or activity observed under normal, physiological conditions (e.g., in a healthy individual without cancer).

As used herein, the term “proliferation” refers to an increase in cell numbers through growth and division of cells.

As used herein, the term “MDM2/MDMX complex” refers to a heterodimer formed by RING domain interaction of the E3 ubiquitin ligases mouse double minute (MDM2)2 and MDMX that mediates polyubiquitination and proteosomal degradation of several proteins, including p53.

As used herein, the term “MDM2/MDMX-dissociating” refers to those compounds, materials, compositions and/or dosage forms, which are capable of dissociating the MDM2/MDMX complex. For example, the MDM2/MDMX-dissociating compound (e.g., Compound 1) described herein dissociates the MDM2/MDMX complex, thereby reducing the amount of the same.

As used herein, the term “reference” refers to a level, expression level, copy number, sample or standard that is used for comparison purposes. For example, a reference sample can be obtained from a healthy individual (e.g., an individual who does not have cancer). A reference level can be the level of expression of one or more reference samples. For example, an average expression (e.g., a mean expression or median expression) among a plurality of individuals (e.g., healthy individuals, or individuals who do not have cancer). In other instances, a reference level can be a predetermined threshold level, e.g., based on functional expression as otherwise determined, e.g., by empirical assays.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “underexpressed” refers to a gene or protein that is expressed or caused to be expressed or produced in a cell at a lower level than is normally expressed in the corresponding wild-type cell. For example, p53 is “underexpressed” in a cancer cell when p53 is present at a lower level in the cancer cell compared to the level in a non-cancerous cell of the same tissue or cell type from the same species or individual. p53 is underexpressed when p53 expression is decreased by 1.1-fold or more (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more) compared to a reference (e.g., a non-cancerous cell of the same type).

As used herein, the term “cancer” refers to a condition characterized by unregulated or abnormal cell growth. The terms “cancer cell,” “tumor cell,” and “tumor” refer to an abnormal cell, mass, or population of cells that result from excessive division that may be malignant or benign and all pre-cancerous and cancerous cells and tissues.

As used herein, the term “p53-associated cancer” refers to a cancer in which p53 is inactivated (e.g., because of dysregulations of a regulatory pathway) or a cancer in which the expression of p53 is decreased compared to a reference (e.g., a non-cancerous cell of the same type). Exemplary p53-associated cancers include breast cancer and B-cell lymphoma.

As used herein, the term “renewable tissues” refers to tissues that are highly sensitive to damage by irradiation, chemotherapeutic drugs and genotoxic agents, and where cell proliferation is important for tissue repair or regeneration. Some examples include bone marrow, spleen, thymus and duodenum.

As used herein, the term “EZH2” refers to the catalytic subunit of the Polycomb Repressive Complex 2 (PRC2) that is responsible for H3K27me3.

As used herein, the term “EZH2-stabilizing” refers to those compounds, materials, compositions and/or dosage forms, which are capable of stabilizing EZH2. For example, the EZH2-stabilizing compound (e.g., Compound 1) described herein stabilizes EZH2 by preventing degradation of the same.

As used herein, “H3K27me3” refers to the tri-methylation of lysine (K)27 sites on histone (H)3 that mediates formation of heterochromatin, and is viewed as a surrogate marker of heterochromatin.

As used herein, “heterochromatinization” refers to compaction and condensation of chromatin, which considerably reduces cellular sensitivity to DNA damage.

### BRIEF DESCRIPTION OF THE DRAWINGS

FIG. 1 is a western blot showing the effect of Compound 1 on the MDM2/MDMX complex.

FIG. 2 is a western blot showing the effect of Compound 1 on ubiquitination and degradation of p53 in MCF-7 cells.

FIG. 3 is a graphical representation of immunohistochemical analyses showing the effect of Compound 1 on expressions of p53, GLUT1, PGK1, Ki67, p21 and PUMA in MCF-7 tumor xenografts.

FIG. 4 is a graph showing the effect of Compound 1 on proliferation of MCF-7 cells.

FIG. 5 is a graph showing the effect of Compound 1 on growth of MCF-7 xenograft tumors.

FIG. 6 is a bar graph showing the effect of Compound 1 on mRNA level of p21 (marker of p53 activity) in MCF-7 cells.

FIG. 7 is a graphical representation of immunohistochemical analyses showing the differential effects of Compound 1 (preserved p53) and Nutlin-3a (activated p53) on caspase-3 activation in thymus of mice treated with the same.

FIG. 8 is a bar graph showing the effect of Compound 1 on mRNA level of ALDHA (marker of HIF1 activity) in MCF-7 cells.

FIG. 9 is western blot and graph showing the effect of Compound 1 on the half-life of EZH2.

FIG. 10 is a western blot showing the effect of Compound 1 on the level of EZH2 and H3K27me3.

FIG. 11 is a bar graph showing the effect of Compound 1 on IR-induced cell death, and the reversal of the effect by GSK126-mediated inhibition of EZH2.

### DETAILED DESCRIPTION

This invention features methods for treatment of cancer in a subject (e.g., a mammalian subject, such as a human) by preserving p53. These methods provide new mechanistic approaches for treating cancer by preserving p53 without its activation, thereby circumventing the systematic toxicity that is caused by p53 activation. Also featured herein are methods of protecting renewable tissues from DNA damage by stabilizing EZH2.

### p53 in Cancer

Dubbed as the “guardian of the genome” and the “cellular gatekeeper”, the p53 protein acts to transmit a variety of stress-inducing signals to different anti-proliferative cellular responses. Hence, p53 can be activated in response to DNA damage, oncogene activation, or hypoxia, in which it subsequently orchestrates biological outputs such as apoptosis, cell-cycle arrest, senescence, or modulation of autophagy. The majority of human cancers acquire mutations that abrogate the p53 tumor suppressor network and, as a consequence, p53 is one of the most extensively studied tumor suppressor proteins in cancer research, with loss-of-function mutations of p53 (mutations that lead to loss of wild-type p53 activity) frequently detected in many different tumor types. p53 functions largely as a transcription factor, and can trigger a variety of anti-proliferative programs by activating or repressing key effector genes. Loss of p53 function occurs in a vast majority of human cancers, representing one of the most important facets in the development and maintenance of malignancies. Perturbations in p53 signaling pathways are believed to be required for the development and progression of most cancers, and there is evidence to suggest that restoration or reactivation of p53 function will have significant therapeutic benefit. Thus, many p53-based strategies have been explored for cancer intervention, including p53 activators, several of which are already in clinical trials. However, while p53 activators have shown clinical efficacy, the resulting toxicities to normal tissues severely limit their use. Hence, there exists a need for optimal modulation of p53 to develop a safe approach for cancer intervention, so as to employ the anti-proliferative and tumor-suppressive functions of p53, while evading the toxic effects associated with p53 activation.

The present invention relates to the identification of an alternative approach for cancer intervention by preserving p53. Using a pharmacological compound, referred to herein as Compound 1, the structure of which is depicted below, a method for preserving p53 without its transcriptional activation was identified.

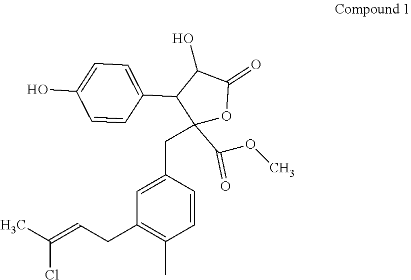 [SMILES: COC(=O)C1(Cc2ccc(C)c(C/C=C(/C)Cl)c2)OC(=O)C(O)C1c1ccc(O)cc1]

Mechanistically, the invention shows that preserved p53 restrains multiple oncogenic transcription factors, which along with their target genes are significantly upregulated across diverse human cancer types where p53 is inactivated. The preserved p53 restrains such oncogenic transcription factors (e.g., HIF1) by directly occupying their genomic target sequences, hence blocking anabolic metabolism and pro-oncogenic signaling pathways. Thus, the present invention discloses new methods for treatment of cancer by preserving p53. Moreover, as preserved p53 in this invention is not activated, the typical systemic toxicity caused by p53 activation is completely avoided, promising a safe therapeutic and prophylactic strategy for cancer intervention.

### MDM2/MDMX Complex

In some embodiments of the invention, the p53-preserving compound preserves p53 by targeting and dissociating the MDM2/MDMX complex. The mouse double minute 2 homolog (MDM2) gene was initially discovered as a p53 binding protein that possesses potent inhibitory effects on p53-mediated transcription. Since then, compelling evidence has emerged for MDM2 to have a physiologically critical role in controlling p53. p53 and MDM2 form an autoregulatory feedback loop. p53 stimulates the expression of MDM2; MDM2, in turn, inhibits p53 activity because it stimulates its degradation in the nucleus and the cytoplasm, blocks its transcriptional activity, and promotes its nuclear export. A broad range of DNA damaging agents induces p53 activation. DNA damage promotes phosphorylation of p53 and MDM2, thereby preventing their interaction and stabilizing p53. Likewise, activated oncogenes sequesters MDM2 into the nucleolus, thus preventing the degradation of p53. Conversely, survival and oncogenic signals mediate nuclear import of MDM2 via Akt activation, which destabilizes p53. Principally, MDM2 is an E3 ubiquitin ligase and promotes p53 degradation through a ubiquitin-dependent pathway on nuclear and cytoplasmic 26S proteasomes. Protein modification by ubiquitin conjugation is an intracellular targeting mechanism, and covalently attached polyubiquitin chains on lysine residues target proteins to proteasomes for degradation. MDM2 is an E3 ligase belonging to the RING family of E3 ligases. MDM2 harbors a self- and p53-specific E3 ubiquitin ligase activity within its evolutionarily conserved COOH terminal RING finger domain (Zinc-binding), and its RING finger is critical for its E3 ligase activity. MDMX (MDM4), is a recently discovered RING finger-containing homolog of MDM2 that associates with MDM2. Genetic studies have shown that MDMX is as essential as MDM2 for negative regulation of p53 during embryonic development. MDMX is not an E3 ligase itself, but cooperates with MDM2 biochemically. In the absence of MDMX, MDM2 is relatively ineffective in repressing p53 because of its extremely short half-life. MDMX renders MDM2 protein sufficiently stable to function at its full potential for gene repression by interacting through their RING finger domains. MDMX shares low overall similarity with MDM2 at the level of amino acid sequence. However, both proteins have a nearly identical RING domain at their C-terminus. A RING domain is a well-established E2-interacting domain that confers E3 ligase activity to RING domain-containing proteins. However, RING domains can also interact with RING domains of other proteins, thus forming protein heterodimers. Interestingly, the MDM2 RING domain was found capable of interacting with the MDMX RING domain, forming a MDM2/MDMX heterodimeric complex, which through the E3 ligase activity of the RING domains, leads to repression of p53. Disruption of this complex is thus a potential way of protecting p53 from being degraded by the E3 ligase activity of the MDM2/MDMX complex, and preserving p53 at its basal physiological level. In some embodiments of the present invention, the p53-preserving compound preserves p53 by dissociating the MDM2/MDMX complex, and preventing MDM2/MDMX-mediated ubiquitination and degradation of p53.

In some embodiments, the compound dissociates the MDM2/MDMX complex, and reduces its amount, e.g., the method includes administering to the subject (e.g., a human subject or animal model) or a cell culture (e.g., a culture generated from a human sample, a cell line, or a repository of human samples) a compound in an amount (e.g., an effective amount) and for a time sufficient to dissociate the MDM2/MDMX complex and reduce its amount. The amount of MDM2/MDMX complex can be decreased in the subject or cell culture at least 1%, 2%, 5%, 10%, 15%, 20%, 25%, 30%, 35%, 40%, 50%, 60%, 70%, 80%, 90%, 95%, 98%, or more, compared to before the administration. The amount of MDM2/MDMX complex can be decreased in the subject or cell culture between 5-20%, between 5-50%, between 10-50%, between 20-70%, between 20-90%.

Although primarily recognized as a regulator of p53, the MDM2/MDMX complex can also regulate and repress other proteins. Among various proteins repressed through this complex, are proteins belonging to the polycomb group (PcG) family, such as enhancer of zeste homolog 2 (EZH2). In some embodiments of the invention, Compound 1, the MDM2/MDMX-dissociating compound protects renewable tissues from DNA damage and cell death by stabilizing EZH2 through disruption of the MDM2/MDMX complex.

### EZH2 and H3K27me3

The PcG family was originally defined in Drosophila. These gene products were grouped together because loss of any of these proteins resulted in a specific homeotic transformation: additional sex combs on male Drosophila legs. The PcG family can be further subdivided into several polycomb repressive complexes (PRCs), among which PRC1 and PRC2 are well characterized. EZH2, a human homolog of the Drosophila PcG protein, E(Z), is the histone lysine N-methyltransferase component and catalytic subunit of the PRC2. EZH2 associates with embryonic ectoderm development (EED) protein and leads to trimethylation of lysine (K)27 of histone (H)3 (H3K27me3). These post-translational histone modifications support a more compact and transcriptionally silent chromatin structure that is more resistant or less sensitive to DNA damage. In some embodiments of the invention, Compound 1, the MDM2/MDMX-dissociating compound protects renewable tissues from DNA damage and cell death by stabilizing EZH2 and EZH2-mediated H3K27me3, which leads to heterochromatinization, rendering the chromatin less sensitive to DNA damage. Thus, the present invention discloses new methods for protecting renewable tissues from DNA damage by stabilizing EZH2 through administration.

In some embodiments, a compound can act to induce or increase EZH2 and EZH2-mediated H3K27me3. One method includes administering to the subject (e.g., a human subject or animal model) or a renewable tissue (e.g., a renewable tissue from a human, or a repository of human tissues) a compound that reduces MDM2-MDMX complex in an amount (e.g., an effective amount) and for a time sufficient to induce or increase EZH2 and EZH2-mediated H3K27me3. EZH2 and EZH2-mediated H3K27me3 can be increased in the subject or renewable tissue at least 1%, 2%, 5%, 10%, 15%, 20%, 25%, 30%, 35%, 40%, 50%, 60%, 70%, 80%, 90%, 95%, 98%, or more, compared to before the administration. EZH2 and EZH2-mediated H3K27me3 can be increased in the subject or renewable tissue between 5-20%, between 5-50%, between 10-50%, between 20-70%, between 20-90%.

In some embodiments, the invention provides therapeutic strategy for protecting normal renewable tissues from DNA damage (e.g., DNA damage caused by radiation and chemotherapeutic drugs) by administration of a compound that reduces MDM2-MDMX complex. For example, methods described in this invention might be extremely beneficial in protecting non-cancerous renewable tissues in a cancer patient who is being exposed to radiation or chemotherapeutic drugs as part of the cancer treatment regimen that leads to collateral DNA damage of normal renewable tissues.

### Cancer

The methods described herein can be used to treat cancer in a subject by administering to the subject an effective amount of a compound that reduces MDM2-MDMX complex. The method may include administering the compound locally (e.g., intratumorally) to the subject in a dose (e.g., an effective amount) and for a time sufficient to treat the cancer. Alternatively, the method may also include administering the compound systemically (e.g., by intravenous infusion) to the subject in a dose (e.g., an effective amount) and for a time sufficient to treat the cancer.

In some embodiments, a compound that reduces MDM2-MDMX complex inhibits or decreases proliferation of cancer cells, e.g., by administering to the subject (e.g., a human subject or animal model) or a cancer cell culture (e.g., a culture generated from a patient tumor sample, a cancer cell line, or a repository of patient samples) a compound that reduces MDM2-MDMX complex in an amount (e.g., an effective amount) and for a time sufficient to inhibit or decrease cancer cell proliferation. Cancer cell proliferation can be decreased in the subject or cancer cell culture at least 1%, 2%, 5%, 10%, 15%, 20%, 25%, 30%, 35%, 40%, 50%, 60%, 70%, 80%, 90%, 95%, 98%, or more, compared to before the administration. Cancer cell proliferation can be decreased in the subject or cancer cell culture between 5-20%, between 5-50%, between 10-50%, between 20-70%, between 20-90%.

In some embodiments, a compound that reduces MDM2-MDMX complex inhibits, delays or decreases growth of tumors. This can be achieved e.g., by administering to the subject (e.g., a human subject or animal model) a compound that reduces MDM2-MDMX complex in an amount (e.g., an effective amount) and fora time sufficient to inhibit, delay or decrease tumor growth. Tumor growth can be delayed or decreased in the subject at least 1%, 2%, 5%, 10%, 15%, 20%, 25%, 30%, 35%, 40%, 50%, 60%, 70%, 80%, 90%, 95%, 98%, or more, compared to before the administration. Tumor growth can be delayed or decreased in the subject between 5-20%, between 5-50%, between 10-50%, between 20-70%, between 20-90%.

A compound that reduces MDM2-MDMX complex can also act to inhibit or decrease cancer cell growth, metastasis, migration, or invasion. Cancer cell growth, metastasis, migration, or invasion can be decreased in the subject or cancer cell culture at least 1%, 2%, 5%, 10%, 15%, 20%, 25%, 30%, 35%, 40%, 50%, 60%, 70%, 80%, 90%, 95%, 98%, or more, compared to before the administration. Cancer cell growth, metastasis, migration, or invasion can be decreased in the subject or cancer cell culture between 5-20%, between 5-50%, between 10-50%, between 20-70%, between 20-90%.

### Cancer Types

In the methods described herein, the cancer may be any solid tumor or hematologic cancer, and may include benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g., head and neck squamous cell cancer); CNS cancer including malignant glioma, astrocytomas, retinoblastomas and brain metastases; malignant mesothelioma; non-metastatic or metastatic breast cancer (e.g., hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

Cancers that can be treated according to the methods described herein include breast cancer, lung cancer, stomach cancer, colon cancer, liver cancer, renal cancer, colorectal cancer, prostate cancer, pancreatic cancer, cervical cancer, anal cancer, vulvar cancer, penile cancer, vaginal cancer, testicular cancer, pelvic cancer, thyroid cancer, uterine cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, a cancer of the central nervous system, a cancer of the respiratory system, and a cancer of the urinary system. Examples of breast cancers include, but are not limited to, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Other cancers that can be treated according to the methods described herein include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leyomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastmic carcinoma, adrenocortical carcinoma, cholangiocarcinoma, Merkel cell carcinoma, DCIS, and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

In some embodiments, the cancer is a p53-associated cancer (e.g., a cancer in which p53 is not mutated but is functionally inactivated). Cancers displaying wild-type p53 include leukemia, lymphoma, breast cancer, prostate cancer, and hepatocellular carcinoma. While the rate of p53 mutation is relatively high in the remaining human cancers, somatic p53 mutations usually occur during the late stage of tumor progression. Therefore, p53 is usually wild-type, but functionally compromised at the early stage of cancer development.

Subjects who can be treated with the methods disclosed herein include subjects who have had one or more tumors resected, received chemotherapy or other pharmacological treatment for the cancer, received radiation therapy, and/or received other therapy for the cancer. Subjects who have not previously been treated for cancer can also be treated with the methods disclosed herein.

The methods described herein can also be used to prevent the development of cancer in a subject (e.g., a human who has not been diagnosed with cancer) by preserving p53 using an effective amount of the p53-preserving compound. The method of preventing the development of cancer by administering the compound may include the steps of: (i) identifying a subject at the risk of developing cancer (e.g., a human subject with mutations of cancer genes including BRCA1 & 2 and other DNA repair genes, a human subject with obesity and/or diabetes, and aged individuals), and (ii) administering to the subject an effective amount of the compound.

### Methods of Treatment

### Formulations and Carriers

This invention describes methods of treating cancer and protecting renewable tissues by administering a compound that reduces MDM2/MDMX. In order to be administered to a subject, a pharmaceutical composition of the compound can be formulated with a pharmaceutically acceptable carrier or excipient. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For oral administration, agents can be formulated by combining the active compounds with pharmaceutically acceptable carriers well known in the art. Such carriers enable the compounds of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

For administration by inhalation, pharmaceutical compositions of this invention may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. For topical application, a pharmaceutical composition may be formulated in a suitable ointment, lotion, gel, or cream containing the active components suspended or dissolved in one or more pharmaceutically acceptable carriers suitable for use in such compositions.

Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

Pharmaceutical compositions may be prepared in microcapsules, such as hydroxylmethylcellulose or gelatin-microcapsule and poly-(methylmethacrylate) microcapsule. Pharmaceutical compositions containing a compound that reduces MDM2/MDMX may also be prepared in other drug delivery systems such as liposomes, albumin microspheres, microemulsions, nano-particles, and nanocapsules. Such techniques are described in Remington: The Science and Practice of Pharmacy 22^{th} edition (2012). The pharmaceutical compositions to be used for in vivo administration must be sterile. This is readily accomplished by filtration through sterile filtration membranes.

### Dosage and Routes of Administration

A compound that reduces MDM2-MDMX complex can be administered as a pharmaceutical composition to a subject (e.g. subject with cancer) in a variety of ways. The composition must be suitable for the subject receiving the treatment, and the mode of administration. A compound used in this invention can be administered orally, sublingually, parenterally, intravenously, subcutaneously, intramedullary, intranasally, as a suppository, using a flash formulation, topically, intradermally, subcutaneously, via pulmonary delivery, via intra-arterial injection, or via a mucosal route.

The dosage of the pharmaceutical compositions of a compound that reduces MDM2-MDMX complex depends on factors including the route of administration, the severity of the condition to be treated, and physical characteristics, e.g., age, weight, general health, of the subject. A pharmaceutical composition may include a dosage ranging from 1 ng/kg to about 100 g/kg (e.g. 1-10 ng/kg, e.g, 2 ng/kg, 3 ng/kg, 4 ng/kg, 5 ng/kg, 6 ng/kg, 7 ng/kg, 8 ng/kg, 9 ng/kg, 10 ng/kg, e.g., 10-100 ng/kg, e.g., 20 ng/kg, 30 ng/kg, 40 ng/kg, 50 ng/kg, 60 ng/kg, 70 ng/kg, 80 ng/kg, 90 ng/kg, 100 ng/kg, e.g., 100-1 μg/kg, e.g., 200 ng/kg, 300 ng/kg, 400 ng/kg, 500 ng/kg, 600 ng/kg, 700 ng/kg, 800 ng/kg, 900 ng/kg, 1 μg/kg, e.g. 1-10 μg/kg, e.g. 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, e.g., 10-100 μg/kg, e.g., 20 μg/kg, 30 μg/kg, 40 μg/kg, 50 μg/kg, 60 μg/kg, 70 μg/kg, 80 μg/kg, 90 μg/kg, 100 μg/kg, e.g., 100-1 mg/kg, e.g., 200 μg/kg, 300 μg/kg, 400 μg/kg, 500 μg/kg, 600 μg/kg, 700 μg/kg, 800 μg/kg, 900 μg/kg, 1 mg/kg, e.g., 1-10 mg/kg, e.g., 2 mg/kg, 3 mg/kg, 4 mg/kg, 5 mg/kg, 6 mg/kg, 7 mg/kg, 8 mg/kg, 9 mg/kg, 10 mg/kg, e.g. 10-100 mg/kg, e.g., 20 mg/kg, 30 mg/kg, 40 mg/kg, 50 mg/kg, 60 mg/kg, 70 mg/kg, 80 mg/kg, 90 mg/kg, 100 mg/kg, e.g., 100-1 g/kg, e.g., 200 mg/kg, 300 mg/kg, 400 mg/kg, 500 mg/kg, 600 mg/kg, 700 mg/kg, 800 mg/kg, 900 mg/kg, 1 g/kg, e.g., 1-10 g/kg, e.g. 2 g/kg, 3 g/kg, 4 g/kg, 5 g/kg, 6 g/kg, 7 g/kg, 8 g/kg, 9 g/kg, 10 g/kg, e.g., 10-100 g/kg, e.g., 20 g/kg, 30 g/kg, 40 g/kg, 50 g/kg, 60 g/kg, 70 g/kg, 80 g/kg, 90 g/kg, 100 g/kg).

The dosage regimen may be determined by the clinical indication being addressed, as well as by various variables (e.g. weight, age, sex of subject) and clinical presentation (e.g. extent or severity of condition). Furthermore, it is understood that all dosages may be continuously given or divided into dosages given per a given time frame. Pharmaceutical compositions that include the p53-preserving compound of the invention may be administered to a subject in need thereof, for example, one or more times (e.g., 1-10 times or more) daily, weekly, biweekly, monthly, bimonthly, quarterly, biannually, annually, or as medically necessary. Dosages may be provided in either a single or multiple dosage regimens. The timing between administrations may decrease as the medical condition improves or increase as the health of the patient declines.

### Combination Therapies

In some embodiments, a compound that reduces MDM2-MDMX complex can be administered in combination with a second therapeutic agent for treatment of cancer. In some embodiments, the second therapeutic agent is selected based on tumor type, tumor tissue of origin, tumor stage, or mutations in genes expressed by the tumor.

### Chemotherapy

One type of therapeutic agent that can be administered in combination with a compound that reduces MDM2-MDMX complex is a chemotherapeutic agent (e.g., a cytotoxic agent or other chemical compound useful in the treatment of cancer). These include anthracyclines (e.g., doxorubicin), nucleoside analogs (e.g., 5-fluorouracil (5-FU)) and related inhibitors, platinum-based anti-neoplastic agents (e.g., cisplatin), taxanes (e.g., paclitaxel), vinca alkaloids (e.g., vincristine), glycopeptide antibiotics (e.g., bleomycin), polypeptide antibiotic (e.g., actinomycin D), alkylating agents, antimetabolites, folic acid analogs, epipodopyyllotoxins, L-asparaginase, topoisomerase inhibitors, interferons, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is leucovorin (LV), irenotecan, oxaliplatin, capecitabine, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art.

### Targeted Therapy

Another type of therapeutic agent that can be administered in combination with a compound that reduces MDM2-MDMX complex is a targeted therapeutic agent. A targeted therapeutic agent blocks the growth of cancer cells by interfering with specific targeted molecules needed for carcinogenesis and tumor growth, rather than by simply interfering with all rapidly dividing cells (e.g., with traditional chemotherapeutic agents). Because most agents for targeted therapy are biopharmaceuticals, the term biologic therapy is sometimes synonymous with targeted therapy when used in the context of cancer therapy (and thus distinguished from chemotherapy, that is, cytotoxic therapy). However, the modalities may be combined to enhance efficacy of the therapy. Targeted therapeutic agents include tyrosine kinase inhibitors, PI3K inhibitors, multi-kinase inhibitors, CDK4/6 inhibitors, mTOR inhibitors, NOTCH inhibitors, HSP90 inhibitors, HSP70 inhibitors, proteasome inhibitors, tumor metabolism inhibitors, janus kinase inhibitors, ALK inhibitors, Bcl-2 inhibitors, VEGFR inhibitors, VEGF inhibitors, and serine/threonine kinase inhibitors among others.

### Cancer Immunotherapy

Another type of therapeutic agent that can be administered in combination with a compound that reduces MDM2-MDMX complex is an immunotherapeutic agent. Immunotherapeutic agents are agents (e.g., drugs, antibodies) that modulate the immune system to treat cancer. The different kinds of immunotherapeutic agents that are currently used in cancer therapy include:

Monoclonal antibodies: man-made versions of immune system proteins that can be very useful in treating cancer as they are designed to attack cancer cells specifically.

Immune checkpoint inhibitors: drugs that take the ‘brakes’ off the immune system, thus helping it to recognize and attack cancer cells.

Cancer vaccines: vaccines that either treat existing cancer or prevent development of a cancer.

Other, non-specific immunotherapies: treatments that boost the immune system in a general way, helping the immune system to attack cancer cells.

In some embodiments, the immunotherapeutic agent that can be administered in combination with a compound that reduces MDM2-MDMX complex is an immune checkpoint inhibitor. Immune checkpoint inhibitors can be broken down into at least four major categories: i) agents such as antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, or KIR), ii) agents such as antibodies that activate stimulatory pathways directly on T cells or NK cells (e.g., antibodies targeting OX40, GITR, or 4-1BB), iii) agents such as antibodies that block a suppressive pathway on immune cells or rely on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, or Ly6G), and iv) agents such as antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, or MUC1). Such agents described herein can be designed and produced, e.g., by conventional methods known in the art (e.g., Templeton, Gene and Cell Therapy, 2015; Green and Sambrook, Molecular Cloning, 2012).

### EXAMPLES

The following examples are put forth so as to provide those of ordinary skill in the art with a description of how the methods described herein may be used and evaluated, and are intended to be purely exemplary of the invention and are not intended to limit the scope of what the inventors regard as their invention.

### Example 1. Dissociation of MDM2/MDMX Complex by Compound 1 Preserves p53

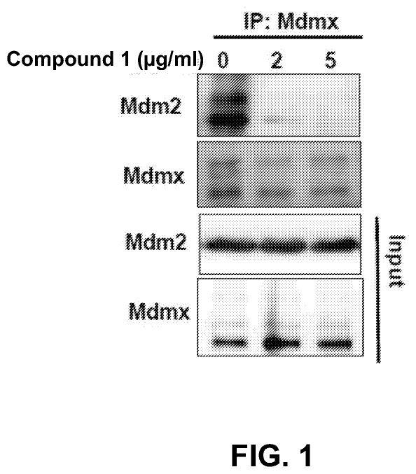

A small molecule compound, referred to herein as Compound 1 was used to dissociate and reduce the MDM2/MDMX complex, and preserve p53. The effect of the compound on MDM2/MDMX complex was validated using a cell-based mammalian two-hybrid assay that revealed a dose-dependent disassociation of the MDM2/MDMX complex by Compound 1. To further verify this finding, splenocytes from wild-type mice were treated with 2 or 5 μg/ml of Compound 1, or vehicle for 24 hour. Immunoprecipitation with anti-Mdmx antibody followed by immunoblot showed disassociation of the MDM2/MDMX complex by Compound 1 (FIG. 1).

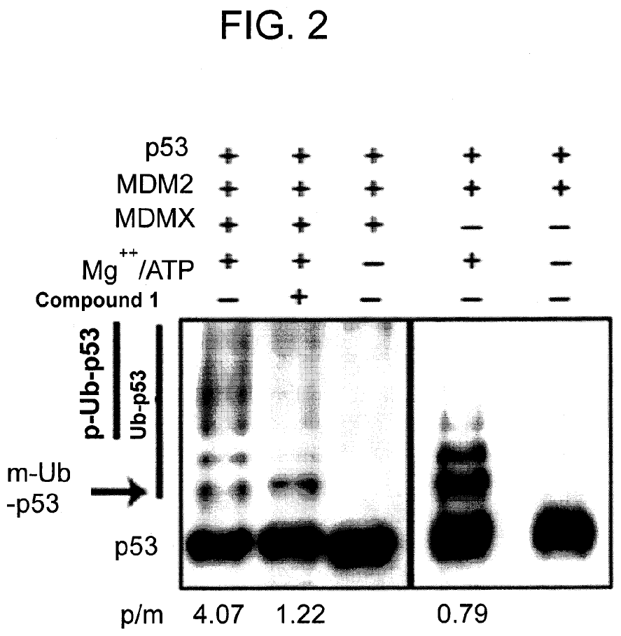

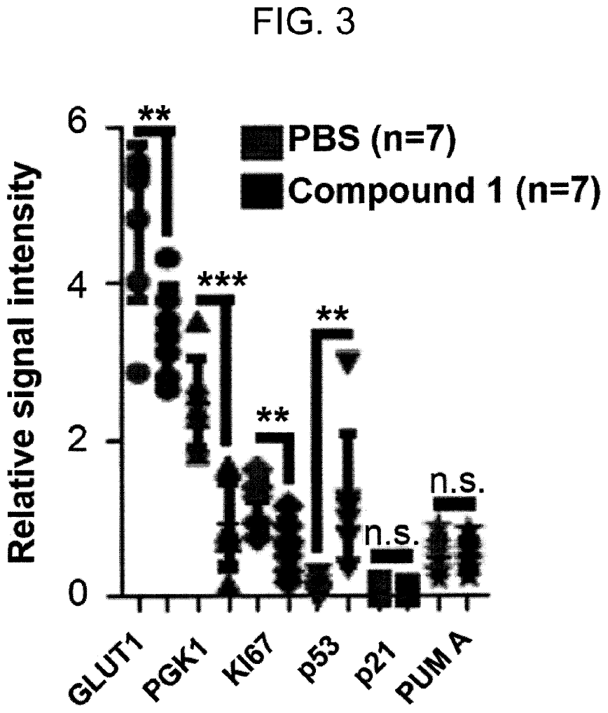

A cell-free assay with purified proteins showed that Compound 1 preserves p53 by blocking MDM2/MDMX-mediated p53 ubiquitination and degradation (FIG. 2). To confirm the finding in vivo, mice xenotransplanted with human breast cancer cells MCF-7 were treated with Compound 1 at a dose of 10 mg/kg intraperitoneally (IP) daily. Immunohistochemical analysis revealed that relative to vehicle-treated mice, tumors isolated from Compound 1-treated mice had readily detectable levels of p53 (FIG. 3), confirming the p53 preserving effects of Compound 1.

### Example 2. Compound 1 Reduces Cell Proliferation

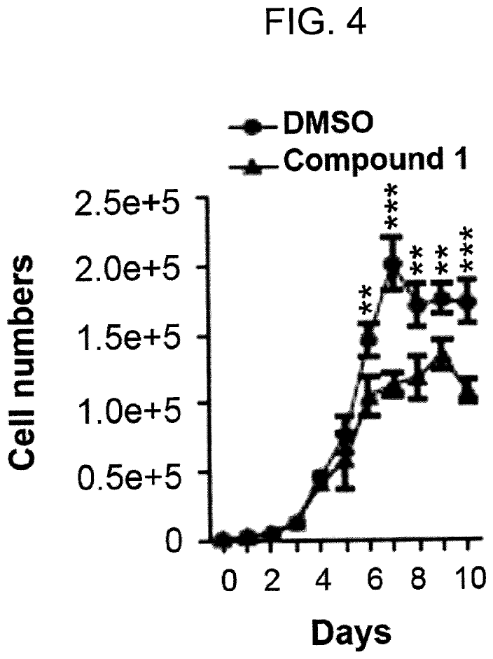

To assess the impact of Compound 1 on cell proliferation, human breast cancer cells MCF-7 were treated with 10 μM of Compound 1. Treatment of MCF-7 cells with Compound 1 resulted in considerable reduction in cell proliferation (FIG. 4). To confirm the finding in vivo, mice xenotransplanted with human breast cancer cells MCF-7 were treated with Compound 1 at a dose of 10 mg/kg IP daily. Immunohistochemical analysis revealed that relative to vehicle-treated mice, tumors isolated from Compound 1-treated mice had readily detectable levels of p53 that was associated with lower level of the proliferation marker Ki-67 (FIG. 3), confirming that Compound 1 preserves p53 to inhibit cell proliferation.

### Example 3. Compound 1 Delays Tumor Growth

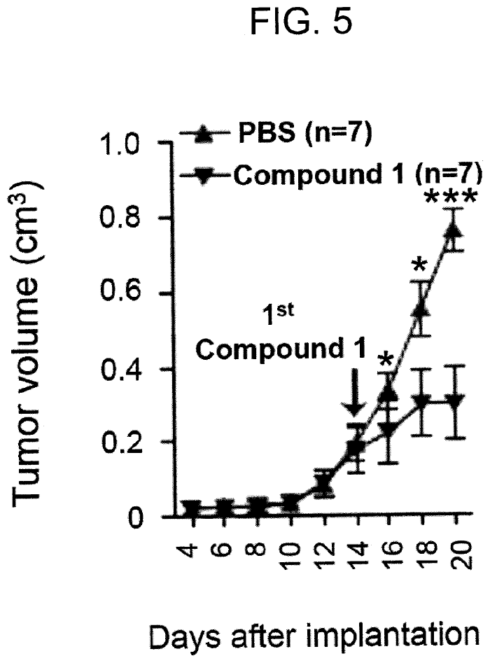

To assess the impact of Compound 1 and preserved p53 on tumor growth, mice xenotransplanted with human breast cancer cells MCF-7 were treated with Compound 1 at a dose of 10 mg/kg IP daily. When compared to vehicle-treated mice, the Compound 1-treated mice showed significant growth delay of tumors derived from MCF-7 cells (FIG. 5). Also, immunohistochemical analysis of tumors revealed that relative to vehicle-treated mice, tumors isolated from Compound 1-treated mice had readily detectable levels of p53 that was associated with lower level of the glycolytic markers GLUT1 and PGK1 (FIG. 3). Expression of GLUT1 and PGK1 serves as markers of aggressive biological behavior and identifies a worse prognosis in breast cancer patients. Hence, reduced level of GLUT1 and PGK1 in Compound 1-treated xenograft tumors indicate that Compound 1 preserves p53 to suppress tumorigenesis and improve survival.

### Example 4. Compound 1 Does Not Induce Apoptosis

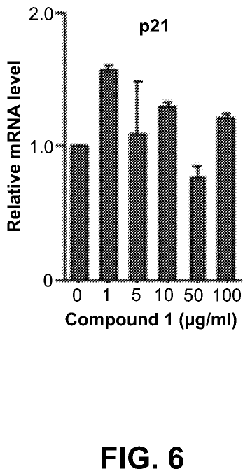

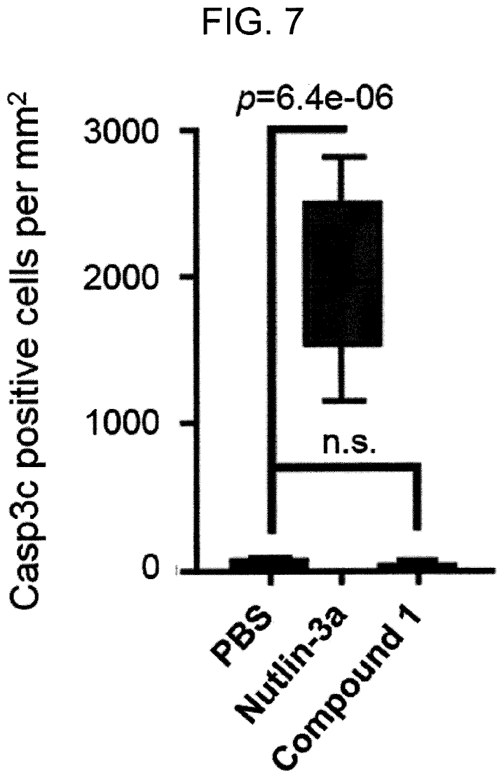

To assess the impact of Compound 1 and preserved p53 on apoptosis, human breast cancer cells MCF-7 were treated with increasing doses (0-100 μg/ml) of Compound 1. RT-qPCR analysis using primers for CDKN1A/p21 showed little induction of p21 (FIG. 6). The expression level of the pro-apoptotic gene p21 was used as surrogate marker to assess the activity of p53. Hence, little induction of p21 upon treatment with Compound 1 indicates that Compound 1 does not activate p53, and does not induce p53-mediated apoptosis. To corroborate this finding in vivo, mice xenotransplanted with human breast cancer cells MCF-7 were treated with Compound 1 at a dose of 10 mg/kg IP daily. Immunohistochemical analysis revealed that relative to vehicle-treated mice, tumors isolated from Compound 1-treated mice had readily detectable levels of p53, associated with little to no change in the expression of pro-apoptotic molecules p21 and PUMA (FIG. 3). To further substantiate these results, an acute toxicity experiment was performed, using Nutlin 3A, a known p53 activator, as a control. C57BL/6 mice were treated with, PBS, Nutlin 3A (200 mg/kg) or Compound 1 (200 mg/kg). The mice were sacrificed 24 hours later, and caspase-3 expression was assessed in thymus, a tissue very sensitive to p53-induced apoptosis. Immunohistochemical analysis revealed that treatment of mice with Nutlin 3A (200 mg/kg) induced considerable apoptosis, as indicated by activated caspase-3. Whereas, treatment with Compound 1 at a dose of 200 mg/kg caused little caspase activation (FIG. 7), indicating that unlike Nutlin3A (which activates p53), Compound 1 (which preserves p53 without activating it) does not induce apoptosis.

### Example 5. Compound 1 Decreases HIF1 Activity

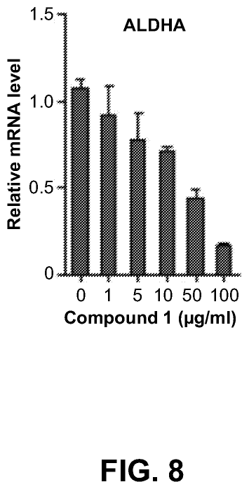

To assess the impact of Compound 1 and preserved p53 on activity of transcription factors (e.g., HIF1), human breast cancer cells MCF-7 were treated with increasing doses (0-100 μg/ml) of Compound 1. The expression level of ALDHA was used as surrogate marker to assess the activity of HIF1. RT-qPCR analysis using primers for ALDOA T showed significant decrease in ALDHA (FIG. 8), suggesting that Compound 1 preserves p53 to restrain and reduce activity of oncogenic transcription factors such as HIF1.

Together these examples implicate that dissociation of MDM2/MDMX complex by Compound preserves p53, resulting in restrained activity of oncogenic transcription factors, reduced cell proliferation, decreased tumorigenesis, and improved survival.

### Example 6. Compound 1 Protects Renewable Tissues

To assess the protective effects of Compound 1 on renewable tissues, wild-type mice were injected with Compound 1 at a dose of 4 mg/kg IP for 19 hours before 4 Gy ionizing radiation (IR) or exposure to doxorubicin (20 mg/kg). TUNEL analysis of thymus, spleen and duodenum showed that pre-treatment with Compound 1 substantially diminished IR- and doxorubicin-induced apoptosis in all the three sensitive renewable tissues (Table 1). This indicated that Compound 1 was capable of protecting renewable tissues from genotoxic stress.

TABLE 1
Values from TUNEL analysis showing the effect of Compound 1
on IR- and doxorubicin-induced apoptosis in renewable tissues

| | Control | IR | | Doxorubicin | |
|---|---|---|---|---|---|
| Renewable | Compound 1: | Compound 1: | Compound 1: | Compound 1: | Compound 1: |
| tissue | 4 mg/kg | 0 mg/kg | 4 mg/kg | 0 mg/kg | 4 mg/kg |
| Thymus | 0% | 41 ± 5.4% | 22 ± 4% | 15 ± 0.1% | 1 ± 0.2% |
| Spleen | 0% | 45 ± 6.1% | 7 ± 0.2% | 22 ± 0.2% | 1 ± 0.1% |
| Duodenum | 0% | 5 ± 0.1% | 1 ± 0.3% | 15 ± 1.1% | 3 ± 0.1% |

### Example 7. Compound 1 Induces EZH2 and EZH2-Mediated H3K27me3

As MDM2 is known to physically interact with EZH2, the MDM2/MDMX complex could potentially be functioning as an E3 ligase to target EZH2 for ubiquitination/degradation. EZH2, the catalytic subunit of PRC2, is responsible for H3K27me3. Alteration of chromatin architecture by H3K27me3-mediated condensation of chromatin and formation of heterochromatin is known to considerably modulate cellular sensitivity to DNA damage. Considering the significance of EZH2 and EZH2-mediated H3K27me3 in cellular protection, the effect of Compound 1 on the same was assessed.

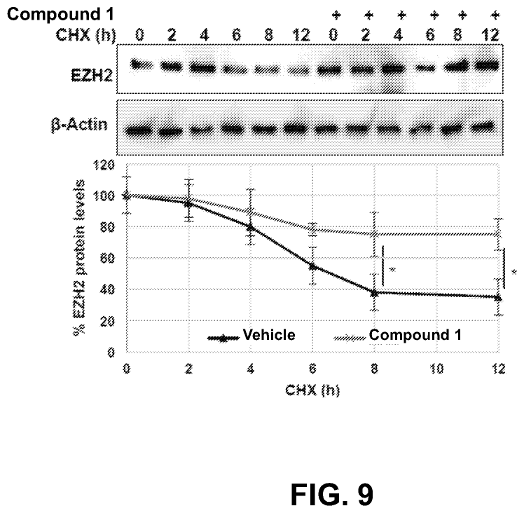

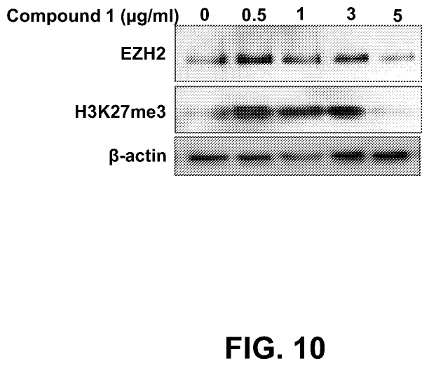

Half-life of EZH2 was measured in splenocytes isolated from wildtype mouse that had been pretreated with 2 μg/ml of Compound 1 for 24 hours followed by 100 μg/ml cycloheximide (CHX). Splenocytes isolated from mice pretreated with Compound 1 showed prolonged half-life of EZH2 (FIG. 9). To further confirm the effect of Compound 1 on EZH2 and H3K27me3, thymocytes isolated from wildtype mice were treated with 0, 0.5, 1, 3 or 5 μg/ml of Compound 1 for 12 hours. Western blot analysis of lysates showed significantly induced levels of EZH2 and H3K27me3 in Compound 1-treated cells (FIG. 10). Furthermore, to validate the effect of Compound 1 on EZH2 and H3K27me3 in renewable tissues, wildtype mice were treated with 0 or 4 mg/kg of Compound 1 for 19 hours. Immunostaining revealed induction of EZH2 and H3K27me3 in thymus, spleen and duodenum of mice who had been pretreated with Compound 1.

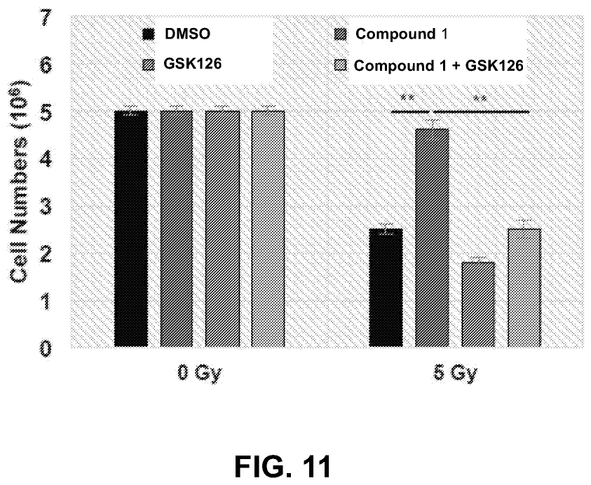

Moreover, the protective effects of Compound 1 were found to be completely reversed in the presence of EZH2 inhibitor, GSK126. Thymocytes isolated from wildtype mice were treated with 2 μg/ml of Compound 1 for 24 hours, in absence or presence of 2 μM GSK126, followed by 0 Gy or 5 Gy IR. Cell numbers were counted 48 hours after IR to assess the effect of Compound 1 and EZH2 inhibition on IR-induced killing. While pretreatment of cells with Compound 1 significantly attenuated IR-induced cell death, the protective effect was reversed by GSK126-mediated inhibition of EZH2 (FIG. 11), indicating that the protective effect of Compound 1 is mediated through EZH2.

Together these examples implicate that Compound 1, through inhibition of MDM2/MDMX-mediated EZH2 degradation has the potential to protect renewable tissues from radiation and genotoxic agent-induced acute injury.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of preserving p53 in a cell without activation, the method comprising contacting the cell with an effective amount of a compound that reduces the amount of MDM2/MDMX complex.

2. A method of preserving p53 in a subject without activation, the method comprising administering to the subject an effective amount of a compound that reduces the amount of MDM2/MDMX complex.

3. A method of treating a subject with cancer, the method comprising administering to the subject an effective amount of a compound that reduces the amount of MDM2/MDMX complex, wherein the reduction of the MDM2/MDMX complex results in preservation of p53 without activation.

4. The method of claim 3, wherein the compound decreases tumor volume, decreases tumor or cancer cell growth, decreases tumor or cancer cell proliferation, preserves tumor or cancer cell p53 activity, and preserves tumor or cancer cell p53 expression.

5. The method of any one of claims 1-3, wherein p53 is preserved without its transcriptional activation.

6. The method of any one of claims 1-3, wherein p53 is preserved without its systemic activation.

7. The method of any one of claims 1-3, wherein the reduction in the amount of MDM2/MDMX complex results from dissociation of the MDM2/MDMX complex.

8. The method of any one of claims 1-3, wherein the compound does not induce apoptosis.

9. The method of any one of claim 3-8, wherein the method further comprises administering a second therapeutic agent

10. The method of claim 9, wherein the second therapeutic agent is an anti-cancer agent.

11. The method of claim 10, wherein the anti-cancer agent is a chemotherapeutic agent.

12. The method of claim 11, wherein the chemotherapeutic agent is an anthracycline.

13. The method of claim 12, wherein the anthracycline is doxorubicin.

14. The method of claim 11, wherein the chemotherapeutic agent is a nucleoside analog.

15. The method of claim 14 wherein the nucleoside analog is fluorouracil.

16. The method of claim 11, wherein the chemotherapeutic agent is a platinum-based anti-neoplastic agent.

17. The method of claim 16, wherein the platinum-based anti-neoplastic agent is cisplatin.

18. The method of claim 11, wherein the chemotherapeutic agent is a taxane.

19. The method of claim 18, wherein the taxane is paclitaxel.

20. The method of claim 11, wherein the chemotherapeutic agent is a vinca alkaloid.

21. The method of claim 20, wherein the vinca alkaloid is vincristine.

22. The method of claim 11, wherein the chemotherapeutic agent is a glycopeptide antibiotic.

23. The method of claim 22, wherein the glycopeptide antibiotic is bleomycin.

24. The method of claim 11, wherein the chemotherapeutic agent is a polypeptide antibiotic.

25. The method of claim 24, wherein the polypeptide antibiotic is actinomycin D.

26. The method of claim 10, wherein the anti-cancer agent is a targeted therapeutic agent.

27. The method of claim 26, wherein the targeted therapeutic agent is one or more of a tyrosine kinase inhibitor, a PI3K inhibitor, a multi-kinase inhibitor, a CDK4/6 inhibitor, an mTOR inhibitor, a NOTCH inhibitor, an HSP90 inhibitor, an HSP70 inhibitor, a proteosme inhibitor, or a tumor metabolism inhibitor.

28. The method of claim 10, wherein the anti-cancer agent is an immunotherapeutic agent.

29. The method of claim 28, wherein the immunotherapeutic agent is one or more of an immune checkpoint inhibitor, a monoclonal antibody, a cancer vaccine, an antibody-drug conjugate, or a non-specific immunotherapeutic agent.

30. The method of claim 29, wherein the immune checkpoint inhibitor is one or more of an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

31. The method of any one of claims 3-30, wherein the compound is administered to the subject in an amount sufficient to treat the cancer or tumor, cause remission, reduce tumor volume, reduce tumor or cancer cell growth, reduce tumor or cancer cell proliferation, preserve tumor or cancer cell p53 activity, preserve tumor or cancer cell p53 expression, or improve survival.

32. A method of preventing the development of cancer in a subject, the method comprising identifying a subject at the risk of developing cancer, and administering to the subject an effective amount of a compound that reduces the amount of MDM2/MDMX complex, wherein the reduction of the MDM2/MDMX complex results in the preservation of p53 without activation.

33. The method of claim 32, wherein p53 is preserved without its transcriptional activation.

34. The method of claim 32, wherein p53 is preserved without its systemic activation.

35. The method of claim 32, wherein the reduction in the amount of MDM2/MDMX complex results from dissociation of the MDM2/MDMX complex.

36. The method of claim 32, wherein the subject has reduced p53 expression or activity.

37. The method of any one of claims 3-36, wherein the cancer is a p53-associated cancer.

38. A method of stabilizing EZH2 in renewable tissue, the method comprising contacting the renewable tissue with an effective amount of a compound that reduces the amount of MDM2/MDMX complex.

39. A method of stabilizing EZH2 in renewable tissue in a subject, the method comprising administering to the subject an effective amount of a compound that reduces the amount of MDM2/MDMX complex.

40. The method of claim 38 or 39, wherein the reduction in the amount of MDM2/MDMX complex results from dissociation of the MDM2/MDMX complex.

41. The method of any one of claims 38-40, wherein the renewable tissue is one or more of bone marrow, spleen, thymus, or duodenum.

42. The method of any one of claims 38-41, wherein the stabilized EZH2 protects the renewable tissue from DNA damage.

43. The method of any one of claims 38-42, wherein the DNA damage is caused by radiation or a chemotherapeutic agent.

44. The method of any one of claims 39-43, wherein the subject has cancer.

45. The method of any one of claims 39-44, wherein the subject is being treated with radiation or a chemotherapeutic agent.
